(19) United States
(12) Patent Application Publication
(10) Pub. No.: US 2026/0345026 A1
(43) Pub. Date: Nov. 5, 2026

(54) METHODS AND SYSTEMS TO VISUALIZE TUMORS AND FUNCTIONAL PARENCHYMA USING MAGNETIC RESONANCE IMAGING (MRI) WITH SUPER-PARAMAGNETIC IRON OXIDE NANOPARTICLES (SPION)

(71) Applicant: Allegheny Singer Research Institute, Pittsburgh, PA (US)

(72) Inventors: Alexander Kirichenko, Pittsburgh, PA (US); Danny Lee, Gibsonia, PA (US); Seungjong Oh, Wexford, PA (US)

(21) Appl. No.: 19/099,988

(22) PCT Filed: Oct. 24, 2023
(86) PCT No.: PCT/US2023/077580

§ 371 Date: Jan. 30, 2025

Related U.S. Application Data
(60) Provisional application No. 63/380,670, filed on Oct. 24, 2022.

(51) Int. Cl. A61B 5/055; A61B 5/00; A61B 5/0515; G06T 7/00

(52) U.S. Cl. CPC A61B 5/055; A61B 5/0515; A61B 5/4244; A61B 5/742; G06T 7/0016; G06T 2207/10096; G06T 2207/30056; G06T 2207/30096

(57) ABSTRACT

Methods of characterizing a tumor in a subject are disclosed. The methods include administering a contrast agent comprising superparamagnetic iron oxide nanoparticles (SPION) to a subject. After administering the contrast agent, a magnetic resonance imaging (MRI) system is used to obtain a set of MRI images of a section of the subject. The system quantifies R2* or other relaxation rates of voxels in the MRI images. The system then uses an auto-contouring tool and the relaxation rates to distinguish functional parenchyma from non-functional tissue in the section. The system displays the image on a display in which the functional parenchyma exhibits a visual appearance that differs from a visual appearance of the non-functional tissue.

### RELATED APPLICATIONS AND CLAIM OF PRIORITY

This patent document claims priority to U.S. Provisional Patent Application No. 63/380,670, filed Oct. 24, 2022. The disclosure of the priority application is fully incorporated into this document by reference.

### BACKGROUND

Many systems exist for identifying tumors in patients. For example, to identify a malignancy in a patient's liver, stereotactic body radiation treatment (SBRT) may be used. Liver SBRT planning, and in particular SBRT in which a technetium TC 99m sulfur colloid injection is used in conjunction with single-photon emission computed tomography (SPECT), helps to identify and avoid functional liver parenchyma volume (FLPV) in patients with advanced hepatic cirrhosis, which can lead to low toxicity and high local control. However, several properties of conventional SPECT/CT, such as poor spatial resolution, non-uniformity and/or distortions, can lead to degradation of the image quality. In addition, SBRT requires the patient to receive radiation dosages, which may not be desired or well tolerated by the patient.

Accordingly, improved methods and systems for distinguishing functional tissue from tumors and/or other non-functional tissue are needed. This document describes methods and systems for addressing these and other issues.

### SUMMARY

This document describes the use of magnetic resonance imaging (MRI or MR imaging) with a contrast agent that includes superparamagnetic iron oxide nanoparticles (SPION) as a technique to identify and distinguish tumors, functional parenchyma and/or non-functional parenchyma using MRI with T2- and/or T2*-weighted images (referred to in this document as T2- and T2*-MRI). The T2 and T2* relaxation time, shortened after an intravenous injection of SPION, is measured and utilized to identify a tumor. For example, the method and system may be used to assess the heterogeneity of a patient's liver for: (1) identifying non-functional liver parenchyma (such as tumors, hepatic veins, and scars associated with cirrhosis); (2) identifying functional liver parenchyma; and (3) identifying and distinguishing functional parenchyma from non-functional tissue such as tumors and/or connective and supporting tissue. The methods and systems described in this document also may use this information to guide online adaptive stereotactic body radiation treatment SBRT and/or liver resection during surgery.

This document also describes auto-contouring methods and tools that use T2- and T2*-MRI with SPION to (1) identify tumors as non-functional liver parenchyma, and (2) quantify volumes of functional liver parenchyma for conformal avoidance during liver SBRT and/or for guiding liver resection during surgery.

### BRIEF DESCRIPTION OF THE DRAWINGS

FIG. 1 illustrates a workflow of a first tool for auto-contouring of a tumor and functional liver parenchyma using SPION-enhanced T2*-weighted MRI.

FIG. 2 illustrates a workflow of a second tool for normalized intensity ratios-based auto-contouring of functional liver parenchyma and hepatic tumors using SPION-enhanced T2-weighted MRI.

FIG. 3 illustrates an example of images of a liver in which functional hepatic parenchyma has been distinguished from non-functional hepatic parenchyma using processes described in this document.

FIG. 4 is a block diagram illustrating example elements of an MRI system.

FIG. 5 illustrates example results of auto-contouring for three different patients using methods described in this document.

FIG. 6 illustrates examples of images that may be taken before SPION administration, after SPION administration, and over the course of multiple days of clinical therapy.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such “first” and “second” are used to modify a noun, such use is simply intended to distinguish one item from another, and it is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

Additional terms that are relevant to this disclosure will be defined at the end of this Detailed Description section.

Additional supporting materials, identified as Examples 1 through 6, are provided at the end of this Detailed Description.

This document describes methods of using magnetic resonance imaging (MRI) to identify and distinguish a tumor (optionally along with other non-functional parenchyma) from functional parenchyma in a subject's body. The document describes the methods with examples in which a subject's liver is examined to distinguish a hepatic tumor from functional hepatic parenchyma. In some embodiments, the methods and systems can be used to identify functional liver parenchyma and thus guide a surgeon to avoid those areas, as they may contain hepatic Kupffer cells which play a critical role in maintaining liver functions. However, the methods also can be used to distinguish a tumor from functional tissue of other organs or parts of a subject's body, as well as to distinguish a tumor from other non-functional tissue.

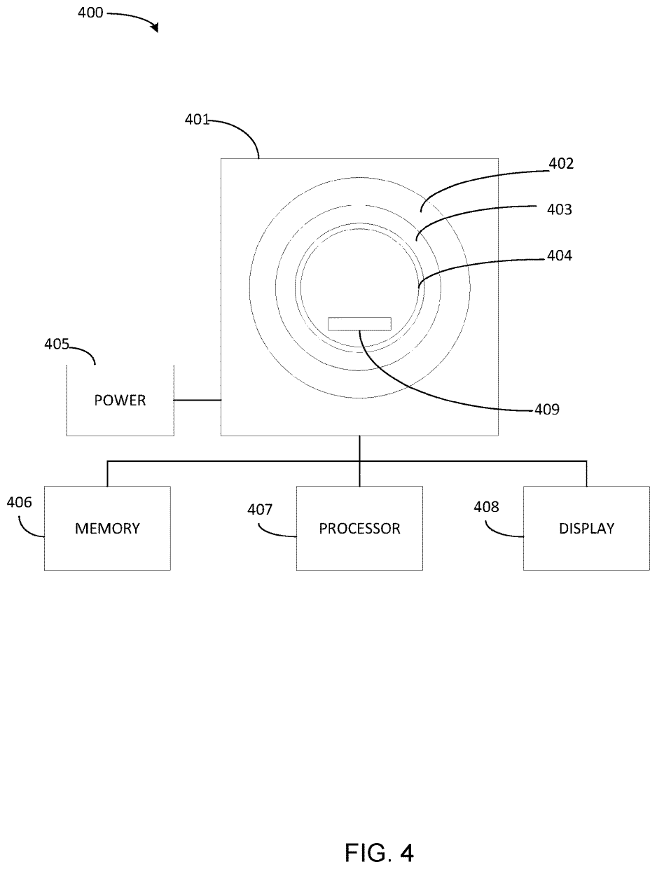

MRI is a non-invasive medical imaging technique that uses a magnetic field and radio frequency (RF) energy to produce high resolution, high contrast images of the organs and tissues inside of the body of a human or other subject. MRI can provide detailed images of soft tissues, abnormal tissues such as tumors, and other structures that cannot be easily imaged using other imaging modalities such as X-rays. Example components of an MRI system will be described below with reference to FIG. 4.

MRI uses nuclear magnetic resonance to visualize the organs, tissues and other internal features of the subject. Contrast agents are often used in MRI to enhance visibility of these features, and the processes described in this document may use such contrast agents. When a subject is positioned in an MRI system, the MRI's magnetic fields cause the hydrogen nuclei of water in the tissues of the imaged region to become polarized in the direction of the magnetic field. The MRI's RF pulses then tip the magnetization generated by the hydrogen nuclei in the direction of the receiver coil, where the nuclei's spin polarization can be detected. The system uses the magnitude of the spin polarization to form an image. However, this magnitude decays back to equilibrium with a characteristic time constant known as the T1 relaxation time. Some contrast agents reduce the T1 relaxation time for protons that are exposed to the agent. The MRI will generate multiple pulses to repeat this process and induce multiple echoes in the nuclei. The MRI detects the echoes and uses their characteristics to generate a set of images.

In addition, the magnetization of the transverse plane will itself decay back to equilibrium over time. This is caused by a phenomenon known as T2 relaxation, which is acquired using a spin echo MRI sequence. While T1 reflects longitudinal relaxation of tissue that appears in the image, T2 is reflects transverse relaxation. For any given MRI image, T2 relaxation time is typically less than the T1 relaxation time.

Any given tissue will exhibit a particular T1 and T2 relaxation time depending on the MRI system and parameters used. However, for any given MRI and parameter set, different types of tissues will have different T1 and T2 relaxation times. Thus, by observing the T1 and T2 relaxation times of various regions of an MRI image, an MRI system can be used to identify different types of tissue based on differences in the T1 and T1 relaxation times that apply to different regions of the image.

Also, the inhomogeneity of the magnetic field in an MRI system induces T2* relaxation, which is acquired during the gradient echo MRI sequence. The T2* relaxation time of an imaged region is typically less than the T2 relaxation time in the imaged region. The amount of T1, T2, and T2* relaxation varies depending on the MRI sequences employed and the contrast agent used. In each case, the variations in T1, T2, or T2* relaxation will alter the contrast of the image.

Examples of such contrast agents that may affect relaxation times include paramagnetic agents (which primarily reduce T1 relaxation time) and ferromagnetic or superparamagnetic agents (which primarily reduce T2 and T2* relaxation time). Example superparamagnetic contrast agents include those made of superparamagnetic iron oxide nanoparticles (SPION or SPIONs) or other ultra-small particles, such as ferucarbotran (including but not limited to that sold under the brand name “Resovist”), ferumoxytol (including but not limited to that sold under the brand name “Feraheme”), and ferumoxtran-10. Other contrast agents include those described in U.S. Pat. No. 5,160,726 to Josephson et al.; U.S. Pat. No. 6,599,498 to Groman et al.; U.S. Patent Application Publication No. 2009/00004113 to Wolf; U.S. Patent Application Publication No. 2003/0225033 to Groman et al.; and U.S. Patent Application Publication No. 2003/0232084 to Groman et al. The relevant disclosures of all of these patents and patent applications are fully incorporated into this document by reference.

In a first process described in this document, a method of using an MRI system to characterize or otherwise identify a tumor and/or functional parenchyma to distinguish the classes of characterized tissue from each other is disclosed. For example, the method may be used on a patient in which a tumor is present on or in the patient's liver. The method may characterize which parts of an MRI image show a tumor, which parts of the image generally show non-functional tissue, and/or which parts of the image correspond to functional parenchyma of the liver (or other organ).

Optionally, as a first step prior to administration of SPION to a subject, an MRI system may be used to capture a set of pre-SPION images of an area of the subject's body in which a tumor is present. A processor may then use the images to quantify R2* relaxation rates of the imaged section.

In the process, SPIONs are administered to the patient. The SPION may be administered by an intravenous therapy, injection, or other process. The SPION will serve as contrast agent in the imaging process. An MRI system is then used to obtain a set of MRI images of the section of the subject's body in which a tumor is present. The administered SPION acts as a contrast agent, to help distinguish the functional tissue from the tumor and/or non-functional tissue in the section of the subject.

After SPIONs are administered to the patient and the MRI captures images of the patient, a processor uses the images to quantify R2* relaxation rates of voxels in the imaged section. R2* is the inverse of T2* (i.e., R2*=1/T2*). In some embodiments, the system could use T2* relaxation rates in instead of R2* relaxation rates. However, because T2* is a very small value, using the inverse provides a value that can more easily be assessed by the tool. The system uses an R2 *-based auto-contouring tool and the R2* relaxation rates to identify the tumor, the functional parenchyma, or both in the images.

Example auto-contouring methods and systems, along with methods of calculating R2* relaxation rates, are described in Example 6 below, which is appended to this disclosure.

To do this, the system determines a threshold such as one using an average of R2* relaxation rates of the section and the tumor.

For example, the threshold may be the average of the R2* of voxels associated with the organ (e.g., Liver-R2*) and the R2* of voxels associated with the tumor (Tumor-R2*), such that:

Threshold=(Liver-R2*+Tumor-R2*)/2.

The threshold may be personalized to a subject by averaging the R2* relaxation rates calculated for that subject, thus enabling an approach to identifying a subject's functional parenchyma that is personalized to the subject. Alternatively, the threshold may be an average of R2* relaxation rates measured across multiple subjects.

The system may then automatically contour the image to distinguish functional parenchyma from the tumor. For example, as explained in Example 6 that is appended to this disclosure, in embodiments where the system has obtained images both prior to and after SPION administration and used both types of images in the R2* relaxation rate calculation, the system may characterize voxels having R2* values that exceed the threshold as representing functional parenchyma, and the system may characterize voxels having R2* values that are below the threshold as representing the tumor. On the other hand, in embodiments where the system has only obtained images after SPION administration, the system may characterize voxels having R2* values that are below the threshold as representing functional parenchyma, and the system may characterize voxels having R2* values that exceed the threshold as representing the tumor.

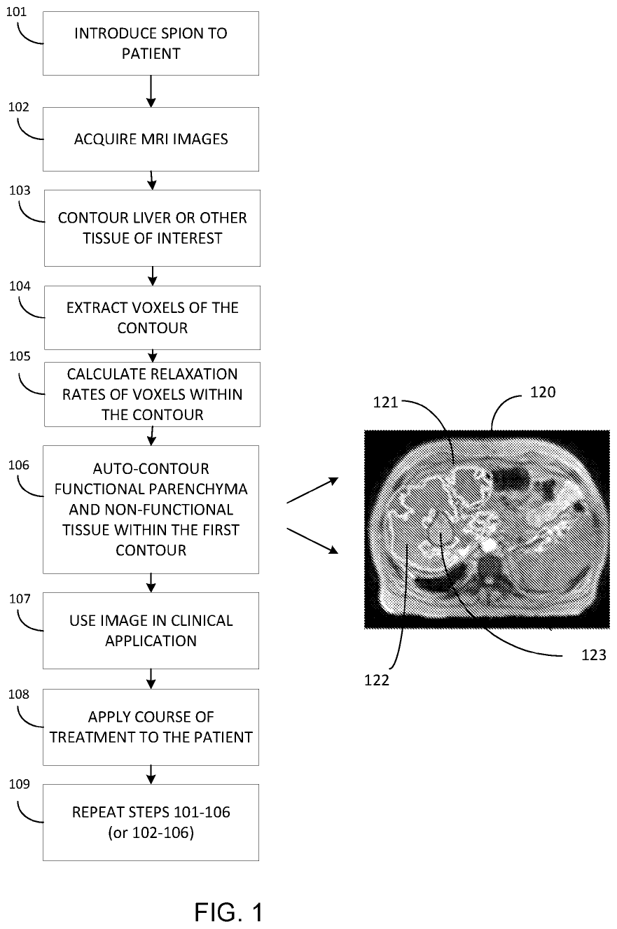

FIG. 1 illustrates a workflow of auto-contouring to identify a tumor in a patient's liver, and to distinguish the tumor from functional liver parenchyma. While this example focuses on the patient's liver, the process can be used for other regions and tissue of a patient's body. In the process, at 101 SPIONs are introduced to the patient, for example by an intravenous (IV) SPION injection. At 102 the system acquires a set of T2*MRI images after SPION injection (post-SPION).

At 103 the system contours the tissue of interest (i.e., the liver) in the post-SPION T2* MRI images. The process of contouring involves drawing a boundary around a region to distinguish one type of tissue from another type of tissue. Processes of contouring are well known in the medical imaging field, and may include manual contouring or automatic (i.e., system-defined) contouring. However, in the present system, the contouring is further refined in the remaining steps of FIG. 1 to distinguish functional parenchyma from non-functional tissue in the liver region.

At 104 the system extracts voxels of the first contour in the image (which in this example is the liver contour determined at 103). At 105 the system calculates relaxation rates of voxels within the first contour, such as voxel-wise R2*. At 106 the system uses auto-contours non-functional tissue and functional liver parenchyma within the first contour based on variations in the R2* between various regions of voxels in the image. This yields an image such as that of image 120, in which the liver region 121 includes functional liver parenchyma 122 and a tumor 123.

At 107 a clinical team or system may use the image in a clinical application by displaying an image that shows the functional liver parenchyma in a color or other visual feature that distinguishes the functional liver parenchyma from the tumor (and optionally other non-functional tissue). (An example of such an image is image 120.) The patient may then be given a course of treatment at 108. The process is repeated at 109 after the treatment to determine whether the tumor has changed in size or otherwise. (The injection of SPIONs may or may not be repeated depending on the time that has passed, as SPIONs can typically remain within a patient for several weeks, and thus SPION injection does not need to be repeated if sufficient SPION levels are still present to serve as a contrast agent.

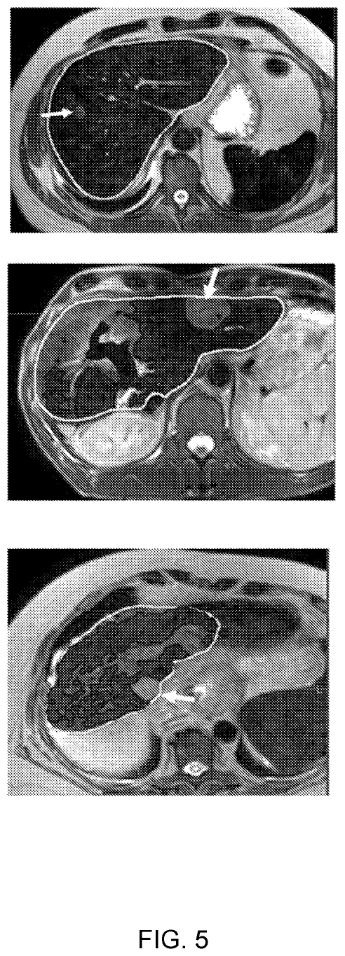

FIG. 5 illustrates example results displayed images after auto-contouring for three different patients using methods described in this document. In the images, the white outline surrounds the region of a liver. The white arrows point to areas in which a metastasis is present; the metastasis is shown in relatively lighter gray while the functional liver parenchyma is shown in relatively darker gray. Image 501 shows a liver with a single metastasis located in the left side of the liver, as indicated by the arrow. Image 502 shows a liver with a single metastasis positioned at the top of the liver. Image 502 shows a liver with a single metastasis positioned at the lower right region of the liver.

The process and results described above provide a visual aid that helps conformal avoidance of Kuppfer cells and other functional parenchyma during targeted radiotherapy treatment, surgery such as hepatectomy, or other clinical applications to assure that an adequate volume of functionally active liver tissue remains after the clinical application.

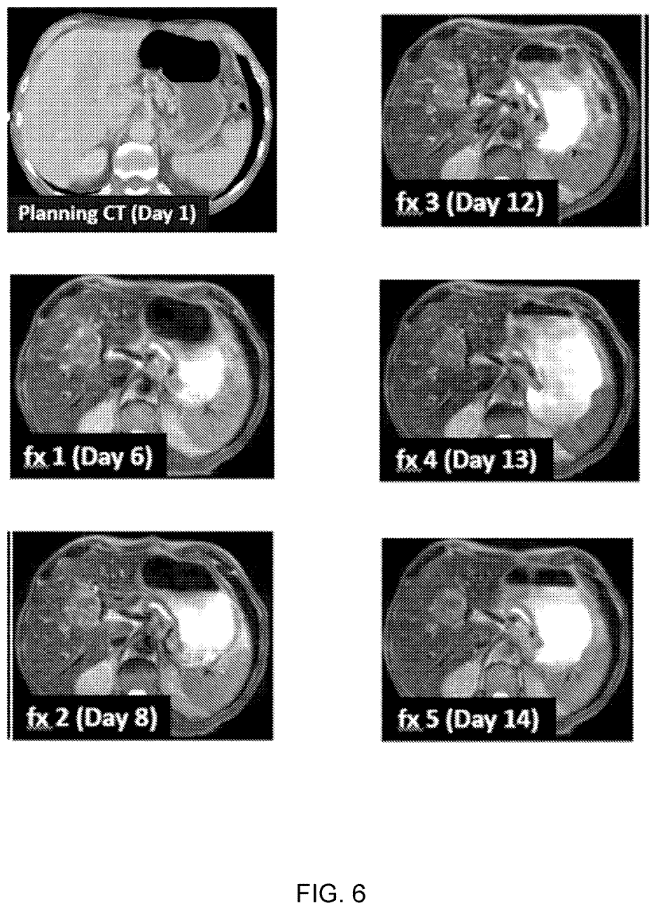

Steps 101 through 106 may be repeated over multiple days, such as over multiple days of targeted radiotherapy treatment, to assess effectiveness of the treatment and quantify reduction in size of the tumor. For example, Example 6 below describes an example in which MRI sessions were two to three days apart before (pre) and after (post) the SPION injection. In each MRI images of a patient were acquired both pre- and post-SPION injection and used for auto-contouring. The patient was then treated with SBRT, after which MRI images of the patient were acquired again. The resulting images allow a clinician to assess tumor and/or functional liver parenchyma volume changes before, during, and after the treatment. FIG. 6 illustrates examples of images that may be taken pre-SPION administration, post-SPION administration, and over the course of multiple days of treatment with SBRT. On the baseline treatment planning CT image, the tumor is shown by the dark region in the top left quadrant of the Day 1, before application of SBRT. MR images taken after a SPION injection and 6 days, 8 days, 12 days, 13 days, and 14 days of treatment show the tumor shrinking in size as the treatment progresses.

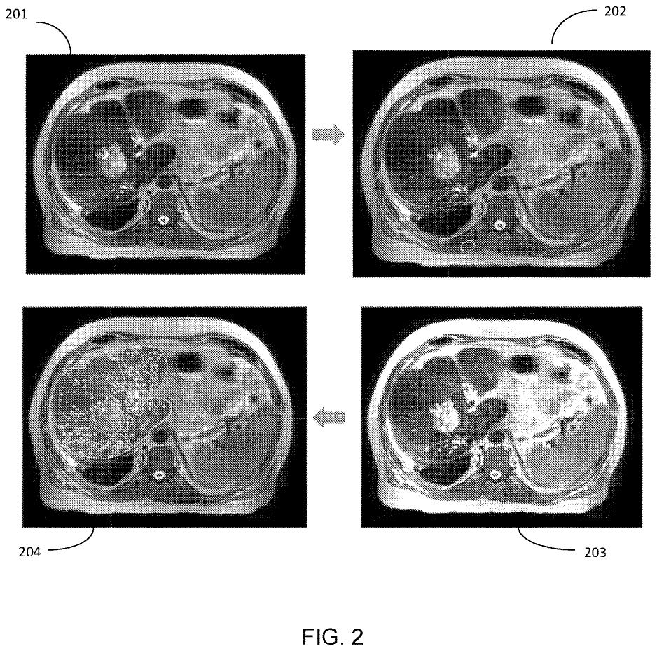

FIG. 2 uses a sequence of MRI images to illustrate a workflow for auto-contouring to identify tumor and functional liver parenchyma using SPION-enhanced, T2-weighted MRI. The process includes at 201 acquiring the T2-weighted image set after the SPION intravenous injection into the patient (post-SPION). At 202 the MRI images from the image set are processed to delineate contours of liver, tumor, and reference regions (in this example, paraspinal muscles) in the post-SPION T2-weighted image set. At 203 the system calculates a normalized image of the post-SPION T2-weighted image set using the magnetic resonance (MR) signal of reference regions. At 204 the system performs auto-contouring on the functional liver parenchyma volume based on the threshold, which is automatically determined using MR signals of the liver and reference regions, using methods such as those described above.

The relaxation times T1, T2, and T2* can be used to determine corresponding relaxation rates, in which R1=1/T1, R2=1/T2, and R2*=1/T2*.

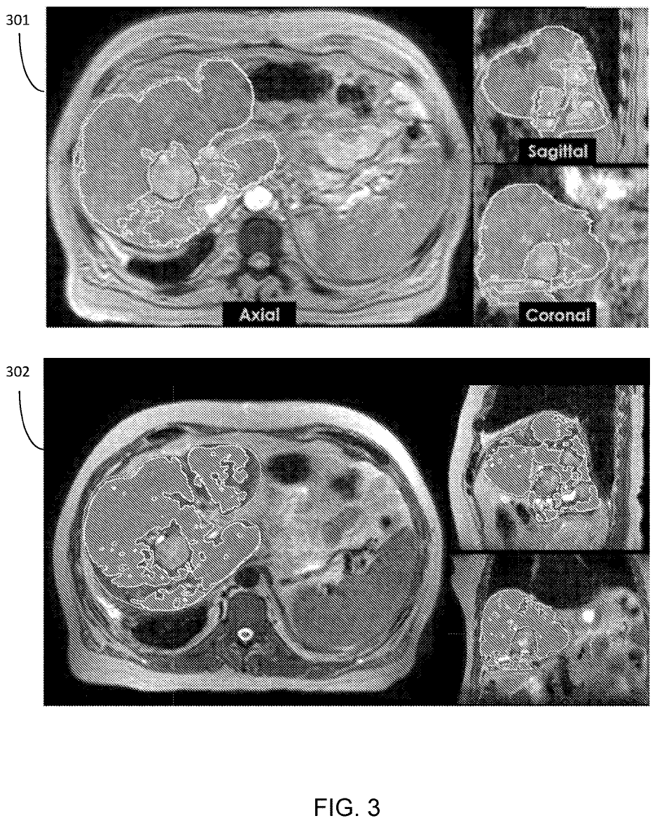

FIGS. 3A and 3B illustrate an example of images of a liver in which functional liver parenchyma have been distinguished from non-functional parenchyma using processes described in this document, and in particular auto-contouring tools using (a) in image 301, R2* relaxation rates and (b) in image 302, a normalized image based on a reference background signal. Areas of functional parenchyma and non-functional liver parenchyma (tumor) are visually distinguished from each other using differently colored borders (i.e., contours) in these images, which if color images were shown may depict for example a yellow border identifying functional parenchyma and a red border identifying the tumor. Other colors may be used.

The characterization methods above may be used during various surgical procedures to identify areas of a tumor that should or should not be extracted during surgery, targeted during radiotherapy, etc. For example, the results may be used to develop a set of radiotherapy treatment parameters during SPION-enhanced 3D-MRI computer-assisted radiotherapy planning to administer stereotactic body radiotherapy (SBRT) to the subject and generate 3D maps of functional and non-functional liver parenchyma for liver resection during surgery. The ability to distinguish functional parenchyma from non-functional parenchyma may help support extraction or targeting of non-functional parenchyma while leaving functional parenchyma undisturbed. This may enable the use of SPION-enhanced MRI for pre-operative surgical planning, for example in advance of complex liver resections to the subject. SPION-enhanced 3D-MRI computer-assisted planning may also support radiation segmentectomy or lobectomy with targeted Y-90 radioembolization to the subject.

Optionally, the MRI system may be one that includes a magnetic resonance imaging guided linear accelerator. If so, the magnetic resonance imaging guided linear accelerator may be also used to administer the SBRT to the subject. The accelerator may apply the functional liver parenchyma, determined by the R2*-based auto-contouring process above, for conformal avoidance of residual hepatic parenchyma and nearby OAR during radiotherapy treatment, preventing post-hepatectomy liver failure after the surgery of liver resection, or other procedures.

FIG. 4 is a block diagram illustrating example elements of an MRI system 400. Illustrated in cross section, an MRI system typically includes a housing 401 with a fixed magnet 402 that will create a strong magnetic field, a gradient coil assembly 403 that enables spatial encoding of signal information, and RF resonators (also known as RF coils) 404 that transmit RF energy to, and receive signals emanating back from, the subject being imaged. An MRI system also includes a power connector 405 for connecting the system to a power source. The MRI system also may include, and/or it be part of a system with a computing device that includes, a processor 406, along with a non-transitory computer-readable memory 407 containing programming instructions that enable the processor to control the system operation and create images from the data that the system obtains. The MRI system also may include or be in communication with a display 408 that will display images that the system captures.

When a subject is positioned inside an MRI system, the subject will lie on a scanner table 409 in a location where the body part to be imaged is centrally located within the opening that is surrounded by the fixed magnet 402, gradient coil assembly 403, and RF coils 404. When the subject is positioned and the system is operated, the magnetic field temporarily realigns water molecules in the subject's body. The RF energy causes these aligned atoms to produce signals that are used to create a two- and three-dimensional, cross-sectional MRI image of the section of the body to which the magnetic field and RF energy are directed.

Early MRI systems were described in U.S. Pat. No. 3,789,832, granted in 1974 to Damadian. Since then, numerous advances have been made to MRI systems, including but not limited to those described in U.S. Pat. No. 8,036,730 to Damadian et al.; U.S. Pat. No. 7,266,406 to Kroeckel; and U.S. Pat. No. 5,168,224 to Maruizumi et al. Combined MRI and radiotherapy systems, along with methods of operating such systems, are described in U.S. Pat. No. 10,945,605, granted in 2021 to Trausch et al; and U.S. Pat. No. 10,806,368, issued in 2020 to Hebert. The disclosures of all of the patents listed in this paragraph are fully incorporated into this document by reference.

The systems described above may include a processor, an imaging device, and a memory containing program instructions. Various embodiments also include computer-implemented methods, performed by a computing device having a processor. Other embodiments include computer program products having programming instructions that, when executed, will cause a processor to perform the methods.

In this document, the terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices. A computer program product is a memory device with programming instructions stored on it.

In this document, the term “imaging device” refers generally to a hardware sensor that is configured to acquire digital images. An imaging device may capture still and/or video images, and optionally may be used for other imagery-related applications. The imaging device may be part of an image capturing system, such as an MRI system, that includes other hardware components. The imaging device can also be mounted on a robotic system, and also may include a transceiver that can send captured digital images to, and receive commands from, other components of the system.

EXAMPLE 1: This example assesses liver function preservation and survival after SBRT with functional treatment planning in patients with hepatocellular carcinoma (HCC) and advanced hepatic cirrhosis. A particular aim of this example was to evaluate treatment outcomes of ablative SBRT with SPECT/CT and MRI-guided treatment planning for localized HCC in patients with Child-Pugh B (CP-B) cirrhosis.

In this example, SPECT with 99mTc-sulfur colloid was co-registered to 3D-CT for identification and avoidance of functional hepatic parenchyma during SBRT in patients with advanced cirrhosis. Likewise, MRI scan using the the Unity MR-Linac (Elekta, Stockholm, Sweden) were used, with a SPION agent as an alternative contrast, for treatment planning and online per-fraction plan adaptations with visualization of tumor and functional liver parenchyma during SBRT.

Functional liver volumes (FLV) obtained from CT-SPECT or SPION-MRI were compared to anatomical liver volumes, as were dosimetric parameters when radiation dose constraints were adapted exclusively to FLV. Hepatic function, toxicity, and radiographic response were documented every 3-4 months following SBRT.

With a median follow-up of 25 months, 44 patients with Child-Pugh B (CP-B) cirrhosis received ablative SBRT to a median dose 48 Gy (3-5 fractions). An automated contouring algorithm was generated for delineation and guided avoidance of best functional hepatic parenchyma on SPION-MRI. Prolonged SPION-contrast retention within hepatic parenchyma allowed per fraction treatment plan adaptation with SPION-enhanced MR imaging of tumor throughout the entire treatment course. Functional volume loss (509 cc or 41.3%, p<0.001) was observed in all patients, while the functional and anatomical liver volumes matched well in a control group of non-cirrhotic/non-HCC patients. While tumors received ablative irradiation, mean dose to residual FLV was maintained well below the liver threshold tolerance to radiation in all patients. Thirty six transplant eligible patients received SBRT as a bridging treatment and 26 patients (60% of the entire cohort) were listed for liver transplant after completion of SBRT. Twenty two patients successfully completed orthotopic liver transplant (OLT) at a median time to transplant of 6.1 months. Eight of 10 patients with intrahepatic progression received additional SBRT with functional treatment planning during follow-up and four of them completed transplant. With 95% in-field local control rate, overall 2-year survival was 63% (90% after the OLT) with no incidence of radiation-induced liver disease (RILD) or Child-Pugh class migration from B to C observed at 6+ months post SBRT.

This example helped to identify that SBRT with functional treatment planning allows identification and guided avoidance of residual functionally active hepatic parenchyma in patients with CP-B cirrhosis. The outcomes reported high local control and low toxicity leading to satisfactory pre- and post-liver transplant outcomes.

EXAMPLE 2: In this example, MR-guided liver SBRT with SPIONs was used to assess primary and metastatic liver tumors during radiotherapy planning and implementation. The process found that SPIONs trapped within cellular compartments of the hepatic Kupffer Cells (KC) produced strong, altering T2 and T2* relaxation effects within hepatic parenchyma infiltrated by KCs while liver tumors with lack of KC appear hyperintense.

In this study, 8 patients with HCC and CP-B cirrhosis or liver metastases completed SPION-MRI simulations on an Elekta Unity MR-Linac and underwent SPION-MRI-guided SBRT to 37.5-50 Gy in 3-5 fractions delivered every other day. An MR image set with navigation was acquired using a T2 3D MR pulse sequence before and after the Ferumoxytol injection. Post SPION-enhanced MR sets were acquired for fast and accurate tumor contouring and sparing FLV during per-fraction online plan adaptation. A custom-built automated contouring algorithm was applied for delineation and guided avoidance of FL V during SPION-MRI guided SBRT. Radiation dose constraints were adapted exclusively to residual FLV. Hepatic function, toxicity, and radiographic response were documented every three months following SBRT. In the study set, radiomodulating properties of Ferumoxytol were assessed on hepatic Kupffer cell lines with phenotyping and Annexin V assay for detection of apoptosis.

In this study, no cytotoxicity or radiosensitizing effects were observed in human monocyte and Kupffer cell lines loaded with Ferumoxytol at clinically relevant concentrations. The study found that SPION facilitates clear and consistent visualization of liver tumors throughout the entire treatment course after a single injection on the day of treatment planning. FLV loss (average 504 cc or 41%, p<0.01) was observed in all patients with HCC and advanced hepatic cirrhosis, while functional and anatomical liver volumes matched well in non-cirrhotic/non-HCC patients. Guided avoidance of FLV during SBRT on SPION-MR-Linac allowed maintaining mean dose to FLV below the liver threshold irradiation. Four patients with HCC were listed for liver transplant after completion of SBRT and one patient successfully underwent an orthotopic liver transplant. With a median follow-up of 6.5 months, all patients in the study maintained in-field local control with no incidence of RILD or Child-Pugh class migration observed after SBRT.

EXAMPLE 3: Functional MRI-Guided SBRT was used on patients with primary liver tumors. As noted above, the inventors have found that liver SBRT planning with 99mTc sulfur colloid SPECT/CT allows identification and avoidance of functional liver parenchyma volume (FLPV) in patients with advanced hepatic cirrhosis, leading to low toxicity and high local control. By using SPIONs as an alternative MR contrast agent, the hepatic parenchyma could be identified on T2 and T2* weighted MR images. The SPION contrast agent (SPION-CA) MR images acquired on the Elekta Unity MR-Linac could be used to visualize the tumor and FLPV, and therefore to guide online adaptive SBRT.

Four patients with hepatic cirrhosis and hepatocellular carcinomas (n=3) and a history of major hepatic resection for cholangiocarcinoma (n=1) completed SPION enhanced MRI guided liver SBRT. All patients underwent two MR simulation sessions to acquire T2 MR image; (1) acquiring pre-SPION T2 image, (2) administering Ferumoxytol as a SPION-CA (i.e., diluted in water for a 2 mg/kg concentration as an IV infusion) for a minimum of 15-20 minutes and (3) acquiring post-SPION T2 image, repeated 48 to 72 hours after the SPION-CA injection. A navigating was unutilized in order to manage breathing motion. On the post-SPION T2 MR image, an MR signal intensity value, and in particular the liver-to-muscle signal intensity ratio (LMR), was calculated based on the average signal intensity of left and right paraspinous muscles. The FLPVs with LMR equal to 0.8, 1.0, 1.2, and 1.4 were automatically segmented and exported for the planning process and online plan adaptation. Radiation dose constraints were adapted exclusively to residual FLPV. FLPV obtained from SPION-MR were compared to anatomical liver volumes obtained on SPECT-CT based FLPV.

The SPION based FLPV using the automated contouring algorithm was efficiently generated and corresponded to the SPECT-CT based FLPV. Various degree of functional volume loss was observed among all patients enrolled in this study, however a major difference was observed in patients with advanced hepatic cirrhosis. While tumors received ablative irradiation, mean dose of residual FLPV was maintained well below the normal liver threshold in all patients. No incidence of radiation liver disease or Child-Pugh class migration was observed at 3+ months post SBRT.

EXAMPLE 4: Ferumoxytol was applied as MRI contrast during adaptive planning on an Elekta Unity MR-Linac. Compared to the non-contrast images, liver tumors after Ferumoxytol injection were superiorly visible for accurate delineation during the entire treatment course. SPIONs are trapped by the hepatic Kupffer cells (KCs) and produce strong T1, T2, and T2* relaxation effects in the liver parenchyma. Malignant tumors with lack of KCs exhibit no signal change, resulting in increased tumor-to-liver contrast difference for MR detection. Once IV injected, SPIONs stay within hepatic KCs for several weeks allowing precise targeting of hepatic tumors with conformal avoidance of residual hepatic parenchyma and nearby OAR. This was the first time in which SPION was recently used for MR-guided liver radiotherapy in a high-field 1.5T MR-Linac.

We investigated two liver cancer patients with primary and metastatic liver malignancies completed MRI simulations and treatments on an Elekta Unity MR-Linac and underwent 5-fraction MR-guided liver SBRT delivered every other day. For liver imaging (Figure A-1 below), a T2 3D TFE (Turbo Field Echo) MR pulse sequence in a 1.5T Unity Philips MR scanner built in the Unity with two MR receiver coils (a 4 channel anterior coil and a 4 channel posterior coil) was used and typical imaging setup parameters were TR/TE=1800/205 ms, FOV=400×400 mm², pixel size=1.56×1.56 mm² and image matrix=256×256, thickness=2.2 mm and flip angle=8°. A navigator window was set at the liver dome scout (⅓ in lung side and ⅔ in liver side) and 233 images were taken in each image set acquired for 250 to 410 seconds. In the first MRI simulation (pre-SPION), an MR image set with navigation was acquired as a baseline followed by injection of SPION contrast agent Ferumoxytol. The second MRI simulation (post-SPION) was repeated at 72 hours after Ferumoxytol injection. Next, a daily MR image set during per-fraction MR-guided liver SBRT was acquired for online treatment plan adaptation. Liver tumors were visually identified to evaluate: (1) the improvement of tumor visualization between pre- and post-SPION image sets, and (2) the consistency of tumor imaging during per-fraction MR image sets.

Compared to pre-SPION image sets, SPION improved tumor visualization on post-SPION images across two liver tumors. Tumor boundary was clearly shown on post-SPION images on the background of negatively enhancing liver parenchyma. During MR-guided liver SBRT, SPION also maintained a consistency of tumor visualization across five per-fraction MR images.

EXAMPLE 5: This study also evaluated the impact of the SPION agent Ferumoxytol on liver tumors and hepatic parenchyma during multi-fractionated online plan adaptations on a 1.5T Elekta Unity MR-Linac.

Eight patients with primary (n=5) and metastatic (n=3) liver malignancies completed Feraheme-enhanced MRI simulations and 3 to 5 fraction SPION-MR-guided liver SBRT delivered every other day. In the first MRI simulation (pre-SPION), an MR image set with navigation was acquired using a T2 3D MR pulse sequence as a baseline followed by an IV injection of Ferumoxytol (i.e., 2 mg/kg). The second MRI simulation (post-SPION) was repeated 72 hours after the injection. Next, plan-MR, verification-MR, and post-MR image sets were acquired for online plan adaptation in each fraction of MR-guided liver SBRT. Liver tumors were visually identified to evaluate: (1) the improvement of tumor visualization between pre- and post-SPION image sets, and (2) the consistency of tumor visualization across multi-fractionated MR image sets.

Compared to pre-SPION T2 image sets, SPION improved tumor visualization on post-SPION T2 image sets in all 8 liver tumors. SPION was only injected once, at 48 to 72 hours prior to the post-SPION MRI simulation in this study. Then, it maintained liver tumor visualization throughout (1) the post-SPION MRI simulation and (2) consecutive 3 to 5 fractionated MR-guided liver SBRT. On pre-SPION T2 image sets, liver tumors were mostly visible however with unclear margins and decreased image sensitivity for smaller lesions in the proximity of vascular structures. On the contrary, liver boundaries were clearly detectable to delineate all liver tumors on post-SPION T2 image sets. Once IV was injected, SPION(s) stayed within hepatic KCs for several weeks allowing precise hepatic tumor targeting with conformal avoidance of residual hepatic parenchyma and nearby OAR(s).

Liver tumor boundaries were clearly visible for delineation of targets and OAR(s) which can (1) minimize delineation uncertainty and shorten treatment planning time, (2) reduce treatment planning target volume, and (3) spare OAR(s) during MR-guided liver SBRT. SPION can facilitate consistent visualization of liver tumors on the day of treatment planning and throughout the entire treatment course, which could be a preferable imaging technique for achieving fast online treatment plan adaptation and accurate MR-guided liver SBRT.

EXAMPLE 6: This process quantified liver heterogeneity via R2*-MRI with SPION to characterize liver functional tissue and tumor tissue.

Kupffer cells (KC) are resident hepatic macrophages and constitute 80-90% of the tissue macrophages present in the whole body. KCs, as one of the major supporting cells, are responsible for maintaining liver function and preventing disease. Therefore, MR imaging that targets resident liver macrophages is often used to identify the volume of functional liver parenchyma (FLPV) using MRI contrast agents.

T2*-MRI, effective transverse relaxation time of T2, may be used as to quantify the magnetic field inhomogeneity of iron deposition in liver. Furthermore, using T2*-MRI with SPION-CA, malignant liver regions become more conspicuous due to susceptibility-related signal loss from SPION uptake with respect to the functional background liver. Similarly, SPION-CA safely labels macrophages for MRI-based cell-tracking, leading to a considerable drop of T2* in the liver. When T2* is transformed into reciprocal R2* (i.e., R2*[Hz]=1000/T2*[ms]), it shows a strong correlation with hepatic iron deposition in the liver as well as the breath-hold gradient echo (GRE)-based 2D and 3D T2*- and R2*-MRI. For these reasons, T2* is increasingly used to image the entire liver in a shorter acquisition time.

Prior to this invention, there was no process that utilized the change of R2* relaxation rates after the SPION injection for quantifying liver heterogeneity to characterize FLP and non-FLP. This process uses SPIONs that enhance liver heterogeneity in the R2* of the liver, and it can be utilized to characterize liver function and tumors while also developing an auto-contouring tool to determine FLPV. The liver image is overlaid with FLPV to evaluate the efficiency of the auto-contouring tool in hopes of protecting healthy tissue during treatment.

In this study, patients with primary and metastatic liver cancers underwent two MRI sessions to acquire T2* image sets before and after the IV injection of a SPION-CA. Voxel-wise R2* relaxation rates in the livers were calculated, and the voxel-wise R2* was analyzed as a function of SPION-enhanced liver heterogeneity to characterize FLP and non-FLP.

This study was comprised of 6 steps, following the process of FIG. 1 of this patent document. First, T2* image sets were acquired before and after the IV SPION-CA injection using a 1.5T Elekta Unity MR-Linac. The two sets of pre- and post-SPION T2* image sets were transferred to MiM software (v7.0.6, MIM Software Inc, Cleveland, OH) for auto-contouring. The liver and tumor contours, manually delineated by a physician for radiation treatment planning, were then copied to both pre- and post-SPION T2* image sets in the MiM. Then, both liver contours of the pre- and post-SPION T2* image sets were extracted by binary masking for the voxel-wise 3D liver volumes. Next, we calculated R2* relaxation rates for R2* liver maps and auto-contoured FLPV. Finally, the FLPV was overlaid on the liver image. Prior to SPION invention, the liver tissue and tumor exhibited similar R2* relaxation rates (a median of approximately 71 Hz). However, at 72 hours after SPION injection the R2* rates of the liver tissue significantly decreased (to a median of approximately 68 Hz), while the R2* rates of the tumor decreased to much lesser extent (to a median of approximately 71 Hz).

Without excluding further possible embodiments, certain example embodiments are summarized in the following clauses:

Clause 1: A method of characterizing a tumor, the method comprising: (a) administering a contrast agent comprising superparamagnetic iron oxide nanoparticles (SPION) to a subject; (b) after administering the contrast agent, using a magnetic resonance imaging (MRI) system to obtain a set of MRI images of a section of the subject, wherein a tumor is present in the section of the subject; (c) quantifying relaxation rates of voxels in the MRI images; (d) using an auto-contouring tool and the relaxation rates to distinguish functional parenchyma from non-functional tissue in the section; and (e) displaying an image on a display in which the functional parenchyma exhibits a visual appearance that differs from a visual appearance of the non-functional tissue.

Clause 2: The method of clause 1, wherein the relaxation rates comprise R2* relaxation rates, and the auto-contouring tool comprises an R2*-based auto-contouring tool.

Clause 3: The method of clause 1 or 2, wherein: (a) the method further comprises obtaining a set of MRI images of the section of the subject prior to administering the contrast agent; (b) quantifying the relaxation rates uses both the MRI images acquired prior to administering the contrast agent and the MRI images acquired after administering the contrast agent; and (c) using the auto-contouring tool and the relaxation rates to distinguish the functional parenchyma from the non-functional tissue in the section comprises: (i) identifying a volume of non-functional tissue as that in which the voxels have relaxation rates that do not exceed a threshold, and (ii) identifying a volume of functional parenchyma as that in which the voxels have relaxation rates that exceed the threshold.

Clause 4: The method of any of clauses 1-3, wherein using the auto-contouring tool and the relaxation rates to distinguish the functional parenchyma from the non-functional tissue in the section comprises: (a) when quantifying the relaxation rates, using only MRI images taken after administering the contrast agent; (b) identifying a volume of non-functional tissue as that in which voxels have relaxation rates that exceed a threshold; and (c) identifying a volume of functional parenchyma as that in which voxels have relaxation rates that do not exceed the threshold.

Clause 5: The method of clause 3 or 4, further comprising determining the threshold by calculating the threshold as the sum of (a) an average R2* in the section (Section-R2*) and (b) an average R2* in the tumor (Tumor-R2*), in each case divided by two such that the threshold equals (Section-R2*+Tumor-R2*)/2.

Clause 6: The method of any of clauses 1-5, wherein the section of the subject comprises a liver of the subject, and the method also comprises using the auto-contouring tool and the relaxation rates to distinguish functional liver parenchyma from the tumor.

Clause 7: The method of any of clauses 1-6, further comprising using a set of radiotherapy treatment parameters during SPION-enhanced 3D-MRI computer-assisted radiotherapy planning to administer stereotactic body radiotherapy (SBRT) to the subject and generate 3D maps of functional and non-functional liver parenchyma for liver resection during surgery.

Clause 8: The method of any of clauses 1-7, further comprising using computer-assisted preoperative surgical planning on SPION-enhanced MRI, for complex liver resections to the subject.

Clause 9: The method of any of clauses 1-8, further comprising using SPION-enhanced 3D-MRI computer-assisted planning for radiation segmentectomy or lobectomy with targeted Y-90 radioembolization to the subject.

Clause 10: The method of any of clauses 1-9, wherein the MRI system comprises a magnetic resonance imaging guided linear accelerator, and the method further comprises using the magnetic resonance imaging guided linear accelerator to administer stereotactic body radiotherapy (SBRT) to the subject.

Clause 11: The method of claim any of clauses 1-10, wherein the method further comprises using the displayed image in a clinical application for conformal avoidance of functional hepatic parenchyma, and the clinical application comprises radiotherapy treatment, or surgery in which the tumor is removed.

Clause 12: The method of claim any of clauses 1-11, wherein the SPION comprises ferumoxytol or ferucarbotran.

Clause 13: A method of characterizing a tumor in a subject, the method comprising: (a) using an MRI system, obtaining a first set of MRI images of a section of the subject during a period of time in which a contrast agent comprising SPION has not been administered to the subject, wherein a tumor is present in the section of the subject; (b) after a first dose of the contrast agent has been administered to the subject and before an additional dose of the contrast agent has been administered to the subject, obtaining a plurality of additional sets of MRI images of the section of the subject; (c) using the additional sets of MRI images and the first set of MRI images to assess characteristics that distinguish the tumor from functional parenchyma; and (d) using the characteristics in a clinical application that treats the tumor while avoiding the functional parenchyma.

Clause 14: The method of clause 13, wherein assessing the characteristics of the tumor comprises determining which parts of the tumor exist in or on non-functional parenchyma of the subject, and which parts of the tumor exist in or on functional parenchyma of the subject.

Clause 15: The method of clause 13 or 14, wherein the clinical application comprises determining a set of radiotherapy treatment parameters to administer SBRT to the subject.

Clause 16: The method of clause 15, wherein the MRI system comprises a magnetic resonance imaging guided linear accelerator, and the magnetic resonance imaging guided linear accelerator is also used to administer the SBRT to the subject.

Clause 17: A method of characterizing a tumor in a subject, the method comprising: (a) using an MRI system to obtain a set of MRI images of a section of the subject after SPIONs have been administered to the subject to provide a contrast agent, wherein a tumor is present in the section of the subject; (b) in the set of MRI images, extracting contours including of a reference region of a body part that is present in the section of the subject; (c) using the reference contour extracted from the set of MRI images, generating MR signal intensity ratios as a normalized image of the body part; and (d) using the normalized image, generating a final image that distinguishes functional parenchyma from the tumor.

Clause 18: The method of clause 17, wherein using the normalized image to generate the final image comprises: (a) identifying a threshold value; (b) classifying voxels in the normalized image having MR signal intensity ratios of body part to the reference contour that do not exceed the threshold value as voxels that show the functional parenchyma; and (c) classifying voxels in the normalized image having MR signal intensity ratios of body part to reference contour that do exceed the threshold value as voxels that show the tumor.

Clause 19: The method of clause 17 or 18, wherein identifying the threshold value comprises calculating average MR signal intensity ratios of the body part to those of the reference contour.

Clause 20: The method of any of clauses 17-19, wherein extracting the contours comprises, by a processor, identifying one or more areas where voxel values do not exceed the threshold value in the normalized image and automatically converting the functional parenchyma volume to contours.

Clause 21: The method of claim any of clauses 17-20, wherein generating the normalized image of the body part comprises normalizing the set of MRI images by the average MR signal intensity of the reference contour.

Clause 22: The method of any of clauses 17-21, wherein the body part is an internal organ of the subject.

Clause 23: The method of any of causes 17-22, wherein the functional parenchyma is that of a liver including Kupffer cells, and the body part comprises paraspinal muscle of the subject.

Clause 24: A computer program product comprising a computer-readable medium with instructions that are configured to cause a processor to perform any of the methods of clauses 1-23.

Clause 25: A system comprising a processor and a computer program product according to any embodiment of clause 24.

Clause 26: The system of clause 27, further comprising an MRI system and a display device, which may be part of the MRI system or separate from the MRI system.

The features and functions described above, as well as alternatives, may be combined into many other different systems or applications. Various alternatives, modifications, variations or improvements may be made by those skilled in the art, each of which is also intended to be encompassed by the disclosed embodiments.

1. A method of characterizing a tumor, the method comprising:
administering a contrast agent comprising superparamagnetic iron oxide nanoparticles (SPION) to a subject;
after administering the contrast agent, using a magnetic resonance imaging (MRI) system to obtain a set of MRI images of a section of the subject, wherein a tumor is present in the section of the subject;
quantifying relaxation rates of voxels in the MRI images;
using an auto-contouring tool and the relaxation rates to distinguish functional parenchyma from non-functional tissue in the section; and
displaying an image on a display in which the functional parenchyma exhibits a visual appearance that differs from a visual appearance of the non-functional tissue.

2. The method of claim 1, wherein:
the relaxation rates comprise R2* relaxation rates; and
the auto-contouring tool comprises an R2*-based auto-contouring tool.

3. The method of claim 1, wherein:
the method further comprises obtaining a set of MRI images of the section of the subject prior to administering the contrast agent;
quantifying the relaxation rates uses both the MRI images acquired prior to administering the contrast agent and the MRI images acquired after administering the contrast agent; and
using the auto-contouring tool and the relaxation rates to distinguish the functional parenchyma from the non-functional tissue in the section comprises:
identifying a volume of non-functional tissue as that in which voxels have relaxation rates that do not exceed a threshold, and
identifying a volume of functional parenchyma as that in which voxels have relaxation rates that exceed the threshold.

4. The method of claim 1, wherein using the auto-contouring tool and the relaxation rates to distinguish the functional parenchyma from the non-functional tissue in the section comprises:
when quantifying the relaxation rates, using only MRI images taken after administering the contrast agent;
identifying a volume of non-functional tissue as that in which voxels have relaxation rates that exceed a threshold; and
identifying a volume of functional parenchyma as that in which voxels have relaxation rates that do not exceed the threshold.

5. The method of claim 4, wherein determining the threshold comprises calculating the threshold as the sum of (a) an average R2* in the section (Section-R2*) and (b) an average R2* in the tumor (Tumor-R2*), in each case divided by two such that the threshold equals (Section-R2*+Tumor-R2*)/2.

6. The method of claim 1, wherein:
the section of the subject comprises a liver of the subject; and
the method also comprises using the auto-contouring tool and the relaxation rates to distinguish functional liver parenchyma from the tumor.

7. The method of claim 1, further comprising using a set of radiotherapy treatment parameters during SPION-enhanced 3D-MRI computer-assisted radiotherapy planning to administer stereotactic body radiotherapy (SBRT) to the subject and generate 3D maps of functional and non-functional liver parenchyma for liver resection during surgery.

8. The method of claim 1, further comprising using computer-assisted preoperative surgical planning on SPION-enhanced MRI, for complex liver resections to the subject.

9. The method of claim 1, further comprising using SPION-enhanced 3D-MRI computer-assisted planning for radiation segmentectomy or lobectomy with targeted Y-90 radioembolization to the subject.

10. The method of claim 1, wherein:
the MRI system comprises a magnetic resonance imaging guided linear accelerator; and
the method further comprises using the magnetic resonance imaging guided linear accelerator to administer stereotactic body radiotherapy (SBRT) to the subject.

11. The method of claim 1, wherein:
the method further comprises using the displayed image in a clinical application for conformal avoidance of functional hepatic parenchyma; and
the clinical application comprises radiotherapy treatment, or surgery in which the tumor is removed.

12. The method of claim 1, wherein the SPION comprises ferumoxytol or ferucarbotran.

13. A method of characterizing a tumor in a subject, the method comprising:
using a magnetic resonance imaging (MRI) system, obtaining a first set of MRI images of a section of the subject during a period of time in which a contrast agent comprising superparamagnetic iron oxide nanoparticles (SPION) has not been administered to the subject, wherein a tumor is present in the section of the subject;
after a first dose of the contrast agent has been administered to the subject and before an additional dose of the contrast agent has been administered to the subject, obtaining a plurality of additional sets of MRI images of the section of the subject;
using the additional sets of MRI images and the first set of MRI images to assess characteristics that distinguish the tumor from functional parenchyma; and
using the characteristics in a clinical application that treats the tumor while avoiding the functional parenchyma.

14. The method of claim 13, wherein assessing the characteristics of the tumor comprises determining:
which parts of the tumor exist in or on non-functional parenchyma of the subject; and
which parts of the tumor exist in or on functional parenchyma of the subject.

15. The method of claim 13, wherein the clinical application comprises determining a set of radiotherapy treatment parameters to administer stereotactic body radiotherapy (SBRT) to the subject.

16. The method of claim 15, wherein:
the MRI system comprises a magnetic resonance imaging guided linear accelerator; and
the magnetic resonance imaging guided linear accelerator is also used to administer the SBRT to the subject.

17. A method of characterizing a tumor in a subject, the method comprising:
using a magnetic resonance imaging (MRI) system, obtaining a set of MRI images of a section of the subject after superparamagnetic iron oxide nanoparticles (SPIONs) have been administered to the subject to provide a contrast agent, wherein a tumor is present in the section of the subject;
in the set of MRI images, extracting contours including of a reference region of a body part that is present in the section of the subject;
using the reference contour extracted from the set of MRI images, generating MR signal intensity ratios as a normalized image of the body part; and
using the normalized image, generating a final image that distinguishes functional parenchyma from the tumor.

18. The method of claim 17, wherein using the normalized image to generate the final image comprises:
identifying a threshold value;
classifying voxels in the normalized image having MR signal intensity ratios of body part to the reference contour that do not exceed the threshold value as voxels that show the functional parenchyma; and
classifying voxels in the normalized image having MR signal intensity ratios of body part to reference contour that do exceed the threshold value as voxels that show the tumor.

19. The method of claim 17, wherein identifying the threshold comprises calculating average MR signal intensity ratios of the body part to those of the reference contour.

20. The method of claim 17, wherein extracting the contours comprises, by a processor, identifying one or more areas where voxel values do not exceed the threshold value in the normalized image and automatically converting the functional parenchyma volume to contours.

21. The method of claim 17, wherein generating the normalized image of the body part comprises normalizing the set of MRI images by average MR signal intensity of the reference contour.

22. The method of claim 17, wherein the body part is an internal organ of the subject.

23. The method of claim 17, wherein the functional parenchyma is that of a liver including Kupffer cells, and the body part comprises paraspinal muscle of the subject.
